(19) United States
(12) Patent Application Publication
(10) Pub. No.: US 2022/0000121 A1
(43) Pub. Date: Jan. 6, 2022

(54) USE OF PENICILLIUM SCLEROTIORUM IN PREPARATION OF PLANT GROWTH REGULATOR OR INDUCER

(71) Applicant: SOUTH CHINA AGRICULTURAL UNIVERSITY, Guangzhou, Guangdong (CN)

(72) Inventors: Jian YAN, Guangzhou (CN); Ping LI, Guangzhou (CN)

(73) Assignee: SOUTH CHINA AGRICULTURAL UNIVERSITY, Guangzhou, Guangdong (CN)

(21) Appl. No.: 17/297,481

(22) PCT Filed: Nov. 7, 2019
(86) PCT No.: PCT/CN2019/116403

§ 371 Date: May 27, 2021

(30) Foreign Application Priority Data
Nov. 28, 2018 (CN) 201811434584.8

(51) Int. Cl. A01N 63/36; A01P 21/00

(52) U.S. Cl. CPC A01N 63/36; A01P 21/00

(57) ABSTRACT

Provided is use of Penicillium sclerotiorum SCAUMCX01, under accession number GDMCC No. 60249, in the preparation of plant growth regulators or inducers. The plant growth regulators or inducers containing Penicillium sclerotiorum, Penicillium sclerotiorum spores, a culture solution thereof and an extract of the culture solution have the effects of promoting the early germination of seeds, promoting the growth of the underground roots of crops and the branching of above-ground parts of crops, improving the bioavailability of crops, delaying the aging progress of crops, and enhancing the growth of crops.

### TECHNICAL FIELD

The present invention relates to the field of agricultural technologies, and in particular, to the use of Penicillium sclerotiorum in the preparation of plant growth regulators or inducers.

### BACKGROUND ART

China is a large agricultural country, and the crop revenue is the major economic source of most agricultural citizens. In order to improve the economic revenue, the common approaches for the agricultural citizens are to increase the amount of fertilizers, and at the same time, spray a large amount of pesticides for reducing the hazards of plant diseases and insect pests, thereby achieving the purpose of increased revenue. However, such operations for a long period of time will lead to soil compaction, and excessive fertilizer and pesticide residues will severely threat people's health. Meanwhile, the ecological environment is further deteriorated.

The plant growth regulator is a class of agricultural agents for regulating the developments and growths of plants, including artificially synthesized compounds having the similar effects as natural plant hormones and the natural plant hormones extracted from organisms. The plant growth regulator is an exogenous non-nutritional chemical substance, and usually can be transport to the action site in the plant, and promotes or suppresses certain aspects of the life process of the plant at a low concentration, so that the plant is developed in the direction that meets the people's demands. However, the effects of the plant growth regulator are affected by various factors, and it is difficult to achieve the optimal effects. For example, climatic conditions, time of administration, drug dosage, method of administration, site of administration, and absorption, transport, integration and metabolism of the crop itself will affect the effects. Therefore, it is important to provide a safe and effective method for reducing the amounts of fertilizers and pesticides.

### SUMMARY

It is an object of the present invention to overcome the disadvantages and deficiencies in the prior art and to provide the use of Penicillium sclerotiorum in the preparation of plant growth regulators or inducers.

The object of the present invention is achieved by the following technical solutions: the use of Penicillium sclerotiorum in the preparation of plant growth regulators or inducers.

The Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249.

The plant is preferably a crop, including rice, corn, wheat, Arabidopsis, and the like.

The plant growth regulator or inducer is the one that promotes the seed germination of the plant, promotes the root growth of the plant, promotes the lateral root increase of the plant, promotes the late stage growth of the plant, improves the bioavailability of the plant, and/or delays the aging progress of the plant.

The major component of the plant growth regulator or inducer is Penicillium sclerotiorum, Penicillium sclerotiorum spores, the culture solution of Penicillium sclerotiorum, the extract of the culture solution of Penicillium sclerotiorum, or the secondary metabolites produced by Penicillium sclerotiorum, wherein and the content of the major component is 0.01 wt %-100 wt %.

The culture solution of Penicillium sclerotiorum is the one obtained by directly cultivating Penicillium sclerotiorum; and it is preferably obtained by the following method: Penicillium sclerotiorum is inoculated in the culture medium, and cultivated for 1-30 days at 100 r/min and at 28° C. so as to obtain the culture solution of Penicillium sclerotiorum.

The cultivation time is preferably 10-14 days; more preferably 14 days.

The medium is PDB medium.

The culture medium of Penicillium sclerotiorum is a solid or liquid medium prepared from a crop, such as rice, rice hull, wheat, wheat hull, potato or the like, as a raw material; preferably Czapek-Dox medium, malt extract medium, or potato medium; more preferably potato medium.

The extract of the culture solution of Penicillium sclerotiorum is the one obtained by extracting with an organic solvent, and the extract may be further concentrated to form a concrete, and then the concrete is formulated into a drug formulation.

The organic solvent is ethyl acetate, chloroform or n-butanol.

The drug formulation may further contain a certain amount of an emulsion agent.

The effective concentration of Penicillium sclerotiorum is 0.0001 mg/ml-10 mg/ml.

Use of Penicillium sclerotiorum as plant growth regulators or inducers.

The Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249.

The use of Penicillium sclerotiorum as plant growth regulators or inducers comprises the culture solution of Penicillium sclerotiorum is irrigated to the plant root, or the culture solution of Penicillium sclerotiorum is sprayed on the plant, or the seeds are soaked into the culture solution of Penicillium sclerotiorum.

The culture solution of Penicillium sclerotiorum is preferably obtained by any one of the following methods:

(1) Penicillium sclerotiorum is inoculated into a culture medium, and cultivated for 1-30 days at 100 r/min and at 28° C. so as to obtain the culture solution of Penicillium sclerotiorum;

(2) Penicillium sclerotiorum is inoculated into a culture medium, and cultivated for 1-30 days at 100 r/min and at 28° C., and then filtered with gauze (removing large bacterial impurity blocks) so as to obtain the culture solution of Penicillium sclerotiorum.

The cultivation time in methods (1) and (2) is preferably 10-14 days; more preferably 14 days.

The culture medium in methods (1) and (2) is PDB medium.

The concentration of the culture solution of Penicillium sclerotiorum is 0.0001 mg/ml-10 mg/ml, and the amount applied to each plant is 1 ml-200 ml (preferably 50 ml).

The plant is preferably a crop, including rice, corn, wheat, Arabidopsis, and the like.

The use of Penicillium sclerotiorum in the preparation of a drug formulation or a biological formulation for improving microorganisms in soil comprises the culture solution containing Penicillium sclerotiorum spores or Penicillium sclerotiorum is applied to promote the growth of the crop or enhance the growth of the crop, and indirectly improve microorganisms in soil, thereby achieving the effects of promoting the adjustment of the growth of the crop or enhancing the growth of the crop.

The Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249

The effective dose of Penicillium sclerotiorum is 0.0005-0.5 wt %.

A method for reducing the application of fertilizers and pesticides comprises the plant growth regulator or inducer is irrigated to the plant root, or the plant growth regulator or inducer is sprayed on the plant, or the plant seeds are soaked into the plant growth regulator or inducer; wherein, the effective ingredient of the plant growth regulator or inducer is Penicillium sclerotiorum, Penicillium sclerotiorum spores, the culture solution of Penicillium sclerotiorum, the extract of the culture solution of Penicillium sclerotiorum, or the secondary metabolite produced by Penicillium sclerotiorum.

The culture solution of Penicillium sclerotiorum is obtained by the following method: Penicillium sclerotiorum is inoculated into the culture medium and cultivated for 1-30 days at 100 r/min and at 28° C. so as to obtain the culture solution of Penicillium sclerotiorum.

The Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249

The plant is preferably a crop, including rice, corn, wheat, Arabidopsis, and the like.

The present invention has the following advantages and effects with respect to the prior art:

1. After investigating on various subjects, it is found in the present invention that the culture solution of Penicillium sclerotiorum can enhance the function of the crop growth, has a wide range of subjects, and has significant effects. The present invention lays a foundation for developing and utilizing Penicillium sclerotiorum to create novel pollution-free plant-derived agricultural agents and growth regulators, and has a good prospect of application and development.

2. It is found in the present application that the drug formulation containing Penicillium sclerotiorum spores, the culture solution thereof, and the extract of the culture solution, alone or in combination with other agents, can provide the effects: promoting the early germination of the seed, promoting the growth of the underground root of the crop and the branching of above-ground part of the crop, improving the bioavailability of the crop, delaying the aging progress of the crop, and enhancing the growth of the crop; therefore the drug formulation can be used as a plant growth regulator and an inducer (the major component is the secondary metabolite produced by Penicillium sclerotiorum or Penicillium sclerotiorum itself).

3. The culture solution of Penicillium sclerotiorum in the present invention can be directly irrigated or sprayed, or concentrated by extraction, and then added with water or other solvent to obtain a formulation at a concentration of 0.0001 mg/ml-10 mg/ml. The above concentration range can achieve the effect of correspondingly adjusting the microbial colonies and activating the plant defense system, thereby obtaining the functions of promoting the growth of the crop and enhancing the resistance of the crop. Alternatively, the Penicillium sclerotiorum spores or the concentrate of the culture solution can be formulated into a biological preparation, with an effective dose content of 0.0005-0.5 wt %.

4. The volume and concentration of Penicillium sclerotiorum spores or the culture solution in the present invention can be adjusted accordingly according to the growth cycle of the crop, and the amount applied to each plant can be 1 ml-200 ml.

5. The culture system of Penicillium sclerotiorum in the present invention may be a solid or a liquid (preferably a liquid). The culture substrate is rice or rice husk, wheat or wheat husk, potato, etc., as the mainly preferred raw material.

### BRIEF DESCRIPTION OF THE DRAWINGS

FIG. 1 is a diagram showing the effect of Penicillium sclerotiorum on corn growth; wherein A) is the result photographed on the day of treating corns with the culture solution or the culture solution containing Penicillium sclerotiorum; B) is the result photographed 1 month after treating corns with the culture solution or the culture solution containing Penicillium sclerotiorum.

FIG. 2 is diagram showing the effect of Penicillium sclerotiorum on the germination of rice seeds; wherein, A) is the result photographed 4 days after treating rice seeds with the pure culture solution or the culture solution containing Penicillium sclerotiorum; B) is the statistical result of germination rate after treating rice seeds with the pure culture solution or the culture solution containing Penicillium sclerotiorum.

FIG. 3 is a diagram showing the effect of Penicillium sclerotiorum on rice growth; wherein, A) is the result photographed on the day of treating rice with the culture solution or the culture solution containing Penicillium sclerotiorum; B) is the result photographed 9 days after treating rice with the pure culture solution or the culture solution containing Penicillium sclerotiorum; and C) is the result photographed 22 days after treating rice with the pure culture solution or the culture solution containing Penicillium sclerotiorum.

FIG. 4 is a diagram showing the effect of Penicillium sclerotiorum on the growth of rice underground roots; wherein, A) is the rice underground root result photographed 22 days after treating rice with the culture solution or the culture solution containing Penicillium sclerotiorum; B) is an average weight statistical plot of rice underground roots.

FIG. 5 is a diagram showing the effect of Penicillium sclerotiorum on the growth of Arabidopsis.

### DETAILED DESCRIPTION OF THE INVENTION

Hereinafter, the present invention will be further described in detail with reference to the examples, but the embodiments of the present invention are not limited thereto. It should be understood that the examples described in this description are merely for illustrating the present invention, and are not intended to limit the present invention. Unless otherwise specified, the agents and raw materials used in the present invention are commercially available.

The Penicillium sclerotiorum in the present invention is obtained from Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249.

The culture medium of Penicillium sclerotiorum in the present invention can be a solid or liquid medium made from the crop, such as rice, rice hull, wheat, wheat hull, potatoes or the like, as raw materials, for example, commercial available Czapek-Dox medium, malt extract medium, potato medium, and the like; preferably a culture medium made from potato as a raw material. The culture medium in the examples is liquid Czapek-Dox medium (PDB), which is purchased from Guangdong Huankai Microbial Sci. & Tech. Co., Ltd.

In the present invention, the culture solution of Penicillium sclerotiorum is obtained by directly cultivating Penicillium sclerotiorum (the cultivated Penicillium sclerotiorum is directly grown on the culture medium). The extract of the culture solution of Penicillium sclerotiorum is obtained by extracting with an organic solvent such as ethyl acetate, chloroform, n-butanol, etc., the extract liquid can be further concentrated and dried to obtain a concrete, the concrete can be further formulated into drug formulation by adding water or a certain amount of emulsion agent, the cultivation time of the culture solution of Penicillium sclerotiorum is 1-30 days (preferably 14 days). The culture solution of Penicillium sclerotiorum can be filtered with gauze so as to remove the large bacterial impurity blocks. In the examples, the method for the preparation of the culture solution containing Penicillium sclerotiorum comprises: Penicillium sclerotiorum is inoculated into the PDB medium, and cultivated for 10 days at the rotation speed of the shaker of 100 r/min, at 28° C. so as to obtain the culture solution containing Penicillium sclerotiorum.

### Example 1

Designed experiment: Corns (B73) (Yan, J. et al. Accumulation of 5-hydroxynorvaline in maize (Zea mays) leaves is induced by insect feeding and abiotic stress, J. Exp. 2015, 66, 593-602) are cultivated to have 3-4 leaves by a conventional method, then 3 pots of plants are used as blank controls (control1, control2, control3), 3 pots of plants are used as test groups (test1, test2, test3); wherein 150 ml of the culture solution containing Penicillium sclerotiorum is directly added into the test groups (3 strains of plants in total, 50 ml per strain of plant), and 150 ml of PDB culture solution is directly added into the blank groups (3 strains of plants in total, 50 ml per stain of plant), and photographs are taken on the same day. Subsequently, water is added in regular amount at regular time every other day to ensure the normal growth of corns. Photographs are taken after 1 month, and used for comparison.

As shown in FIG. 1, it is found that after the culture solution containing Penicillium sclerotiorum is added, the corns grow better, have strong stem, and the leaves thereof at the bottom are aged slowly.

### Example 2

20 rice seeds (Oryza. Sativa L. spp. japonica) (Yan J. et al. The Tyrosine Aminomutase TAM1 Is Required for beta-Tyrosine Biosynthesis in Rice, Plant Cell, 2015, 27, 1265-1278) are respectively placed in blank culture solution (namely, PDB culture solution) and the culture solution containing Penicillium sclerotiorum, and the germination rate of the seeds is observed after 4 days.

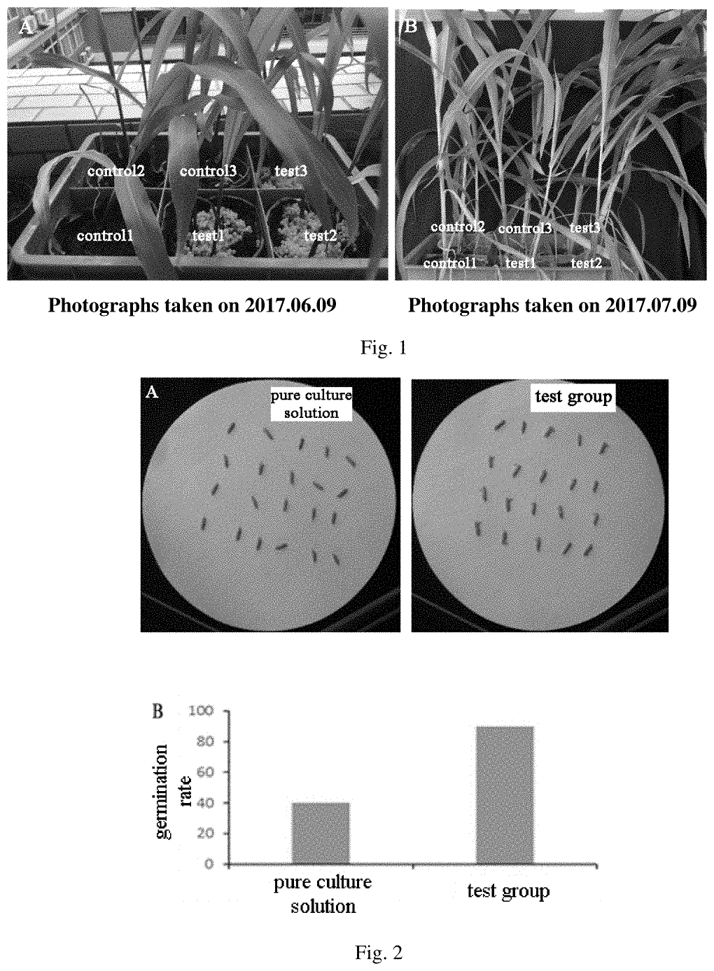

The photograph result is shown in FIG. 2(A), and it is obtained from the statistical result of data (FIG. 2(B)) that the culture solution containing Penicillium sclerotiorum can significantly increase the germination rate of rice seeds.

### Example 3

When rices (Oryza. Sativa L. spp. japonica) (Yan J. et al. The Tyrosine Aminomutase TAM1 Is Required for beta-Tyrosine Biosynthesis in Rice, Plant Cell, 2015, 27, 1265-1278) are grown to have 4-5 leaves, 50 ml of the culture solution containing Penicillium sclerotiorum and the blank culture solution (namely, PDB culture solution) are respectively added to each plant, repeated 3 times, labeled as control group (control1-3) and test group (test1-3) respectively. Photographs are taken on the same day (Jul. 29, 2017), then photographs are taken on Aug. 7, 2017, and Aug. 20, 2017, respectively, and used for comparison.

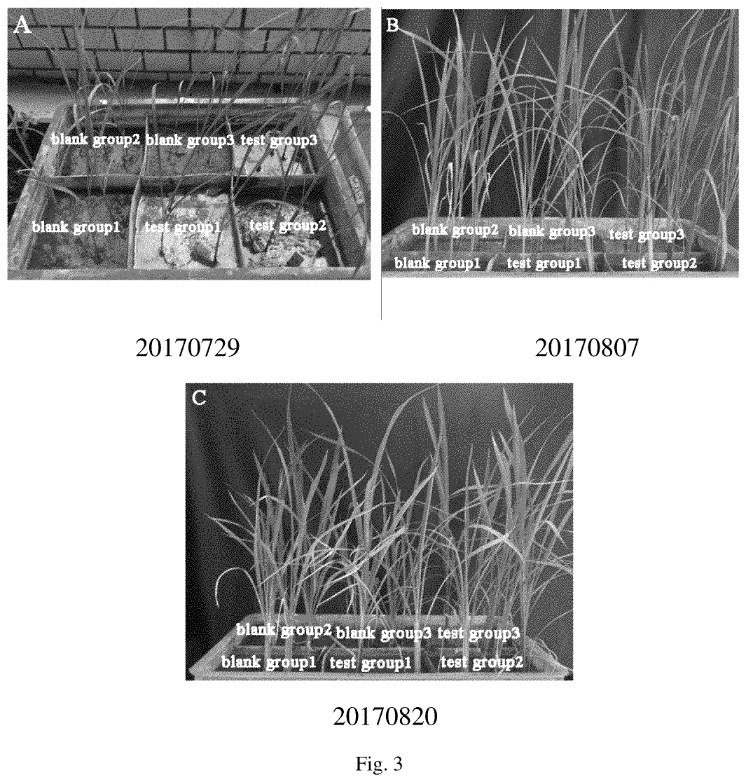

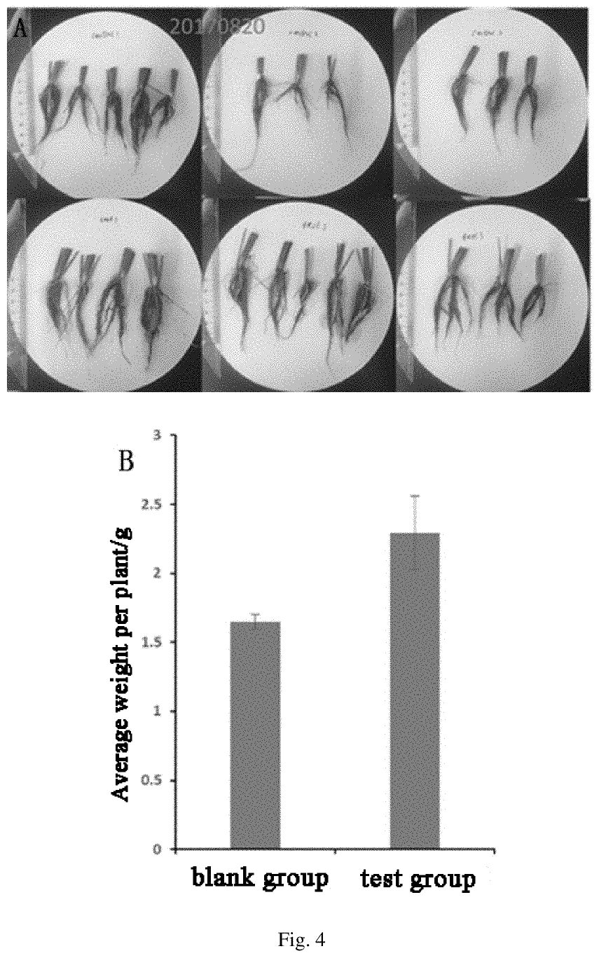

As shown in FIG. 3, it can be easily seen that after the culture medium containing Penicillium sclerotiorum is used, the rices grow stronger and the leaves turn yellow less. The growth of the underground roots is further analyzed, and as shown in FIG. 4, it can be easily seen that the roots of the test groups applied with Penicillium sclerotiorum develop more and grow more. The dry weight statistical result of roots (comparison of Aug. 20, 2017 with the day of treatment) also provides the consistent result—the root weight of the Penicillium sclerotiorum test group increases significantly.

### Example 4

Designed experiment: when Arabidopsis (Arabidopsis thaliana) (http://signal. salk. edu/) is grown to have 4 leaves, 3 pots of Arabidopsis under the same grow status are selected, divided into 3 groups, and then the test group is directly added with 50 ml of the culture solution containing Penicillium sclerotiorum (labeled as test group A), and the blank group is directly added with 50 ml of PDB culture solution (labeled as culture solution-1B) or pure water (labeled as water 1C), and photographs are taken on the same day; wherein, 1 pot of plant is used as water blank control, 1 pot of plant is used as culture solution blank control, and 1 pot of plant is used as test group of culture solution containing Penicillium sclerotiorum. Subsequently, water is added in regular amount at regular time every other day to ensure the normal growth of Arabidopsis, and photographs are taken after 10 days and used for comparison (labeled as test group A1, culture solution-1B1, water-1C1). The above experiments are repeated on the same day by adding 50 ml of culture solution containing Penicillium sclerotiorum, the culture solution and pure water respectively, and photographs are taken after 10 days (test group A2, culture solution-1B2, water-1C2);

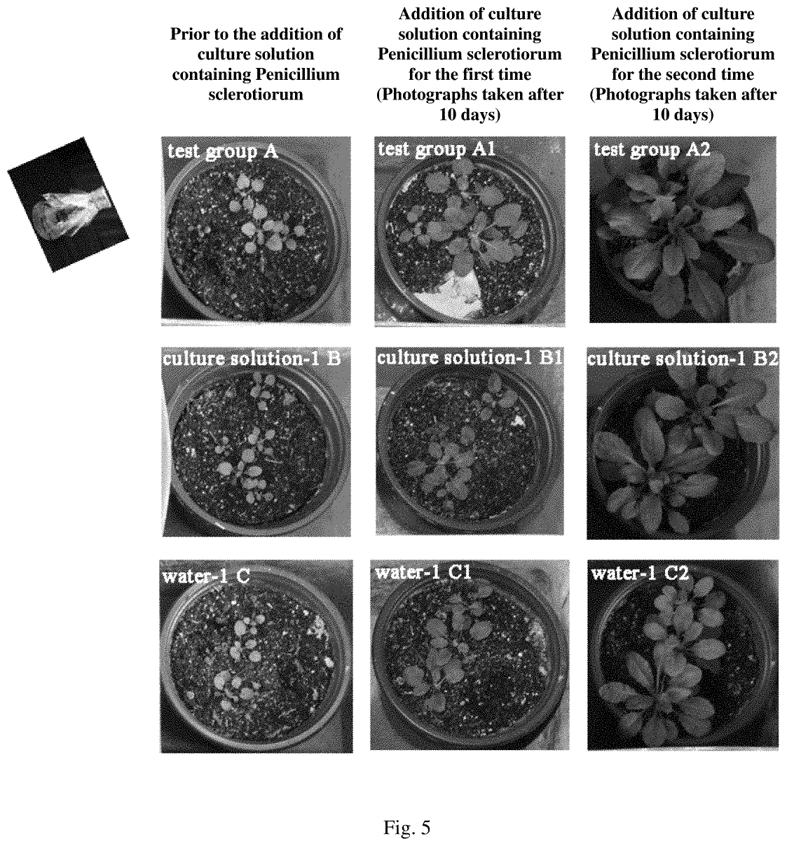

Some of the results are shown in FIG. 5. It was found that after the culture solution containing Penicillium sclerotiorum is added, the Arabidopsis grow better, has larger leaves and more leaves.

The above embodiments are preferred embodiments of the present invention, but the embodiments of the present invention are not limited to the above examples, and any other changes, modifications, substitutions, combinations and simplifications made without departing from the spirit and principle of the present invention, which should be equivalent replacement manners, all fall in the scope of protection of the present invention.

1. Use of Penicillium sclerotiorum in the preparation of plant growth regulators or inducers, characterized in that said Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249.

2. The use of Penicillium sclerotiorum in the preparation of plant growth regulators or inducer according to claim 1, characterized in that:
the plant is a crop;
the effective concentration of Penicillium sclerotiorum is from 0.0001 mg/ml to 10 mg/ml.

3. The use of Penicillium sclerotiorum in the preparation of plant growth regulators or inducer according to claim 2, characterized in that:
the plant is rice, corn, wheat or Arabidopsis.

4. The use of Penicillium sclerotiorum in the preparation of plant growth regulators or inducer according to claim 1, characterized in that:
the plant growth regulator or inducer is the one that promotes the seed germination of the plant, promotes the root growth of the plant, promotes the lateral root increase of the plant, promotes the late stage growth of the plant, improves the bioavailability of the plant, and/or delays the aging progress of the plant.

5. The use of Penicillium sclerotiorum in the preparation of plant growth regulators or inducers according to claim 1, characterized in that:
the major components of the plant growth regulators or inducers are Penicillium sclerotiorum, Penicillium sclerotiorum spores, the culture solution of Penicillium sclerotiorum, the extract of the culture solution of Penicillium sclerotiorum, or the secondary metabolites produced by Penicillium sclerotiorum;
wherein the culture solution of Penicillium sclerotiorum is obtained by the following method:
Penicillium sclerotiorum is inoculated into a culture medium, and cultivated for 1-30 days at 100 r/min and at 28° C. so as to obtain the culture solution of Penicillium sclerotiorum;
wherein the medium is PDB medium.

6. Use of Penicillium sclerotiorum as plant growth regulators or inducers, characterized in that said Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249.

7. Use of Penicillium sclerotiorum as plant growth regulators or inducers according to claim 6, characterized in that the culture solution of Penicillium sclerotiorum is irrigated to the plant root, or the culture solution of Penicillium sclerotiorum is sprayed on the plant, or the seeds are soaked into the culture solution of Penicillium sclerotiorum;
wherein the culture solution of Penicillium sclerotiorum is obtained by any one of the following methods:
(1) Penicillium sclerotiorum is inoculated into a culture medium, and cultivated for 1-30 days at 100 r/min and at 28° C. so as to obtain the culture solution of Penicillium sclerotiorum;
(2) Penicillium sclerotiorum is inoculated into a culture medium, and cultivated for 1-30 days at 100 r/min and at 28° C., and then filtered with gauze so as to obtain the culture solution of Penicillium sclerotiorum;
wherein the culture medium in methods (1) and (2) is PDB medium.

8. Use of Penicillium sclerotiorum as plant growth regulators or inducers according to claim 6, characterized in that:
the plant is a crop;
the concentration of the culture solution of Penicillium sclerotiorum is from 0.0001 mg/ml to 10 mg/ml.

9. Use of Penicillium sclerotiorum in the preparation of drug formulations or biological formulations for improving microorganisms in soil, characterized in that:
said Penicillium sclerotiorum is Penicillium sclerotiorum (from Portulaca oleracea L.) SCAUMCX01, deposited on Oct. 11, 2017 at Guangdong Microbial Culture Collection Center, 5th floor, 59 blg, No. 100, Xianlie Middle Road, Yuexiu District, Guangzhou, Guangdong Province, under accession number GDMCC No. 60249.
wherein the effective dose of Penicillium sclerotiorum is 0.0005-0.5 wt %.

10. The use of Penicillium sclerotiorum in the preparation of plant growth regulators or inducers according to any one of claims 1 to 5, characterized in that the plant growth regulator or inducer is irrigated to the plant root, or the plant growth regulator or inducer is sprayed on the plant, or the plant seeds are soaked into the plant growth regulator or inducer.
